(19) United States
(12) Patent Application Publication
(10) Pub. No.: US 2005/0186288 A1
(43) Pub. Date: Aug. 25, 2005

(54) Compositions and methods for the prophylaxis and treatment of aphthous ulcers and herpes simplex lesions

(75) Inventors: Win L. Chiou, Burr Ridge, IL (US); Linda L. Chiou, Burr Ridge, IL (US)

Correspondence Address: MERCHANT & GOULD PC, P.O. BOX 2903, MINNEAPOLIS, MN, 55402-0903, US

(73) Assignee: Chiou Consulting Inc., Burr Ridge, IL (US)

(21) Appl. No.: 10/999,988

(22) Filed: Dec. 1, 2004

Related U.S. Application Data
(63) Continuation-in-part of application No. PCT/US02/18223, filed on Jun. 7, 2002.

(51) Int. Cl. A61K 33/24; A61K 33/34; A61K 33/26; A61K 33/06

(52) U.S. Cl. 424/617; 424/682; 424/646; 424/630

(57) ABSTRACT

Disclosed is a topical method for providing highly effective analgesic, anti-inflammatory, antimicrobial and tissue-regenerating activities for the hitherto most effective prophylaxis and treatment of aphthous ulcers and herpes simplex lesions and for the effective treatment of burns and other oral mucosal lesions as well as for prophylaxis and treatment of gingivitis, periodontitis and tooth mobility comprising topically administering to the affected tissue an effective amount of a composition comprised of one or more safe and efficacious polyvalent metal compounds such as magnesium sulfate and aluminum potassium sulfate alone or preferably with one or more safe and efficacious anti-inflammatory compounds, such as a novel ultra-low-strength (0.02%) hydrocortisone acetate, that potentiate the activities of polyvalent metal compounds.

### CROSS-REFERENCE TO RELATED APPLICATIONS

This application is a Continuation-In-Part of PCT/US02/18223, filed Jun. 7, 2002, which application is incorporated herein by reference.

### BACKGROUND

Aphthous ulcers, also known as canker sores or ulcerated stomatitis, are a common oral lesion. Approximately 20% of the population will suffer from the annoyance and irritation of a canker sore in their lifetime. (Gastrointestinal Disease: Pathophysiology/Diagnosis/Management, 5th edition, M. H. Sheisenger et al., eds., W.B. Saunders Company, Philadelphia, p. 273 (1992).) Canker sores are generally round, clearly defined, painful, shallow ulcers in the lining of the mouth (oral mucosal surfaces). They usually begin with a tingling or burning sensation (prodromal period), followed by a red spot or bump (a swelling of the tissue) that ulcerates. The ulcers/canker sores are often covered with a grayish-white exudate and surrounded by an erythematous (inflammatory) margin. They range in size from pinpoint (minor aphthae) to over one-quarter inch in diameter (major aphthae). Canker sores usually cause discomfort and/or pain, and they may cause pain so severe that difficulties in speaking and eating result; which can lead to weight loss. (Diseases of the Oral Cavity,” In: Drug Treatment: Principles and Practice of Clinical Pharmacology and Therapeutics,@ G. S. Avery, ed., Aids Press, Sydney, Australia (1976).) Canker sores usually last from about 7 to about 10 days, but may last for several weeks.

The cause of canker sores is unknown, but a number of conditions are believed to be associated with the disorder, suggesting that more than one cause is likely. Some canker sores are thought to be related to an abnormal immune system and are probably genetically influenced. Emotional stress and mouth injury, such as those caused by dental procedures, aggressive tooth cleaning or local trauma, such as when the tongue or cheek is bitten, are thought to trigger outbreaks. Further etiologic factors include immunologic, microbiologic, viral, nutritional factors, such as dietary deficiencies (especially iron, zinc, folic acid or vitamin B₁₂), menstrual periods, hormonal changes and food allergies (to substances in nuts, chocolate, acidic foods (such as vinegar, pickles or citrus) and glutin). (Oral Pathology: Clinical Pathologic Correlations, 3rd edition, J. A. Regezi et al., eds., W.B. Saunders Company, Philadelphia, pp. 46-53 (1999).)

Currently, there is no cure for canker sores. However, there are several palliative treatments that can soothe the sores. Commonly suggested treatments include topical antiseptics, antibiotics and anesthetics (such as 2% viscous lidocaine or 10-20% benzocaine gel) to temporarily relieve pain. Topical application of high-potency synthetic glucocorticoids such as triamcinolone acetonide (0.1%) and dexamethasone (0.1%) has also been used (Oral and Maxillofacial Pathology by B. W. Neville et al., pp. 236-239 (1995)). Because of their potential serious adverse effects, the use of such glucocorticoids requires a prescription.

Recently, amlexanox oral paste 5% (Aphthasol7 from Block Drug Company) was made available as a prescription drug to treat aphthous ulcers. However, its efficacy is still quite limited. For example, after 4 days (four times daily) of continuous treatment, complete resolution of pain and complete healing of ulcers was achieved in only 60% and 37% of patients, respectively.

U.S. Pat. No. 5,981,499 by Hau discloses a topical method to deliver a supratherapeutic level of antibiotic, the sole essential ingredient, to shallow aphthous ulcers to inhibit opportunistic pathogenic bacteria in the oral mucosa that is substantially higher than levels achieved by oral, intramuscular or intravenous administration of antibiotic. In the method disclosed by Hau, the medicament is in the form of a powder or troche and includes a dry dosage of antibiotic such as one of the known penicillins, beta-lactam antibiotics, tetracyclines, aminoglycosides, cephalosporins, macroglides, vancomycin, bacitracin, chloramphenicol and their salts and mixtures thereof. Hau further discloses that the powder or troche may also include an effective amount of a water-insoluble salt or oxide of a polyvalent metal compound, a non-essential, optional, supplemental ingredient, such as magnesium, zinc, calcium, aluminum, bismuth, titanium, and copper and mixtures thereof to form a protective barrier in a suspension (ABSTRACT, column 4, lines 8-16; column 6, lines 38-44) over the aphthous ulcer. Hau discloses that under such an alternative combined approach, the presence of the antibiotic prevents most microorganisms of the normal flora from surviving or multiplying, permitting tissue regeneration processes to occur under the protective barrier coating. Hau emphasizes that control of infection is essential for promoting the healing process. As disclosed by Hau, the troche or powder may be mechanically directed in place, for example, by the fingers. Once in contact with the aphthous ulcer, the troche or powder may be held in position by the tongue for about 5 to 15 minutes. Hau discloses that painful symptoms improve 24 hours after treatment, ulcers display visible signs of healing within 2 days, and canker sores heal in about two to four days. After treatment aphthous ulcers are known to re-occur.

However, the methods disclosed by Hau are limited in several ways. For example, it can be difficult or impossible to hold the dosage form for 5 to 15 minutes in hard-to-access areas of the mouth. Additionally, administration can be difficult or impossible in most infants and small children. Another serious drawback of the methods disclosed by Hau are that some patients may be allergic to antibiotics. Also, the development of antibiotic-resistant microorganisms can occur as a result of the methods of Hau.

Another type of common lesion is caused by the herpes simplex virus (HSV) (Neville, B. W. et al., Oral & Maxillofacial Pathology, W.B. Saunders Company, Philadelphia, 181-186 (1995)). There are two types of herpes simplex virus, HSV-1 and HSV-2. Symptoms of HSV-1 infection occur mainly in the pharynx, intraoral sites, lips, eyes and skin above the waist, while those of HSV-2 occur mainly on the genitalia and the skin below the waist. The most common recurring site for HSV-1 is the vermilion border and adjacent skin of the lips, known as herpes labialis (“cold sore” or “fever blister”). An oral HSV outbreak occurs in about 15 to about 45 percent of the population (Neville et al., p. 183). Usually, from about 6 to about 24 hours before the lesions develop, prodromal symptoms appear (such as pain, burning, itching, tingling, a localized warmth and erythema of the involved area; these symptoms being referred to as “pre-ulceration” or “prelesion” symptoms of herpes simplex lesion herein). Multiple small, erythematous papules may develop and form clusters of vesicles. The vesicles may rupture and crust in about 2 days. Abolishment of pain and healing of ulcer, or lesion usually occurs within about 7 to about 10 days.

Currently, there are no commercial drug products that can effectively treat or cure herpes simplex lesions. Topical pastes or gels containing certain specific analgesics such as benzocaine or benzyl alcohol have been commonly available as over-the-counter products to temporarily relieve the pain of the disease. Abreva® (docosanol), a topical over-the-counter product marketed by GlaxoSmithKline Pharmaceutical Company, was reported to perform only slightly better than the placebo (mean healing time of 4.1 days versus 4.7 days for placebo in one study) in treating cold sores (The Wall Street Journal, Jan. 19, 2001). Prescription products containing topical antiviral drugs, such as Denavir® (penciclovir cream), were also reported to be of limited value in treating herpes simplex lesions (The Wall Street Journal, Jan. 19, 2001). Silver nitrate, now abandoned due to its toxicity (no claims for silver compounds are made in this invention), has been earlier used for treating aphthous ulcers.

Thus, current topical treatments for aphthous ulcers or herpes simplex lesions generally require several applications a day and take several days to abolish the pain and inflammation and/or heal the ulcer/lesion. Additionally, there are no known effective topical treatments for prevention of the formation of ulcers/lesions (e.g., prophylactic). Thus, there is currently a need for an effective treatment of lesions, including herpes simplex lesions and aphthous ulcers. Specifically, there is a need to develop an effective, inexpensive, simple, safe, rapid, convenient, and preferably nonprescription-required method to prevent and treat such lesions.

### SUMMARY

By simply employing a non-essential, optional, supplemental ingredient in Hau's teaching such as magnesium sulfate in a mucoadhesive paste, we have surprisingly discovered that this simple, commonly available ingredient alone can achieve therapeutic effect/result/benefit in the treatment of aphthous ulcers exceedingly much more superior than those reported by Hau; pain stops almost immediately after application and complete healing of ulcers is achieved within about 12 to about 48 hours after treatment. Furthermore, the magnesium sulfate used is extremely water-soluble and will not form a physical barrier taught by Hau. Therefore, this approach is totally departing from or even directly contradicting the most fundamental teachings of Hau. This surprising therapeutic result is accomplished even without the use of any conventional analgesic, anesthetic, anti-inflammatory compound or anti-microbial compound. We further surprisingly discovered that polyvalent metal compound(s) alone can effectively treat (eliminate) various prodromal or pre-ulceration symptoms (burning sensation, pain, irritation, or red bump or spot) of aphthous ulcers thereby preventing subsequent outbreak of more painful, more uncomfortable and longer-lasting aphthous ulcers. We also surprisingly discovered that the least-toxic (low-potency) and safest glucocorticoids, namely, the endogenous hydrocortisone and cortisone as well as their acetate or other derivatives, which have been commonly or universally ignored in the last half a century as effective compounds for treating or preventing aphthous ulcers (no textbooks and no drug companies advocate their use in this respect) is found to be effective for treating pre-ulceration symptoms or for preventing and treating aphthous ulcers even at “super-low” concentrations (e.g., 0.02% or perhaps even much lower, such as 0.001%, the solubility of hydrocortisone acetate in water) compared to the 0.1% strength of high-potency glucocorticoids. Moreover, combination of a polyvalent metal compound (such as 0.5% to 2.0% magnesium sulfate) and an anti-inflammatory compound such as a glucocorticoidal compound (such as 0.05% hydrocortisone acetate) surprisingly has substantial synergistic effect in treating pre-ulceration symptoms or in preventing and treating aphthous ulcers; extremely efficacious results such as almost complete and immediate relief of pain and complete cure (not recurrent after discontinuation of treatment) of prodromal symptoms or ulcers can be achieved within about 12 to 48 hours of treatment, thus representing an unprecedented, revolutionary advance in drug therapy. The above efficacious, surprising findings and methods of treatments are not simply predictable or “extrapolated” from the teaching of prior arts of Hau and others without actual experimentation.

It is to be emphasized that a zinc or magnesium salt (zinc or magnesium pentosan polysulfate) alone is shown to be ineffective in treating herpes infection (wound or ulcer) in spite of the wound-healing property of the zinc or magnesium (U.S. Pat. No. 5,514,667 by D. Cullis-Hill). Furthermore, during the pre-ulceration or prodromal period of aphthous ulcers or herpes simplex, there is no wound to be healed or to be cured by a wound-healing compound and there is no need for tissue regeneration; this aspect is completely not addressed by Hau.

Furthermore, since the pain associated with the pre-ulceration symptoms or aphthous ulcers can be almost immediately stopped from the present invention, and patients can practically and immediately assume normal functions such as eating, drinking, talking and sleeping without any significant distraction or discomfort, the present invention may be regarded to practically provide an immediate cure or relief for the suffering from the oral lesion even though the healing of the lesion is not completed; this is the case especially when no flare-up occurs. Thus, the present invention represents an unprecedented major advance in the prophylaxis and treatment of aphthous ulcers.

Polyvalent metal compounds alone described in the present invention are surprisingly potentially effective in the prophylaxis and treatment of herpes simplex. This aspect is also completely not addressed by Hau. In sharp contrast with the treatment of aphthous ulcers, high-potency glucocorticoidal compounds, known to have a wound-healing effect, are contraindicated in the treatment of herpes simplex lesions (Handbook of Nonprescription Drugs, 13^{th} edition, American Pharmaceutical Association, Washington, D.C., 2000, page 669), even though the ulcerative lesions from both diseases are similar.

Because of the present surprising discovery of the unique combined synergistic properties of analgesic, anti-inflammatory, anti-microbial and tissue-regenerating effects of polyvalent metal compounds, these compounds can also be useful to treat or prevent gingivitis, periodontitis, and tooth mobility. This may represent an important alternative to commonly employed antibiotic therapy for gingivitis and periodontitis. For tooth mobility (a common dental problem) there is currently no drug therapy available and it is generally considered as an irreversible disease. Thus, the present discovery may represent an important breakthrough for reversing tooth mobility problem. A combination with one or more effective anti-inflammatory compounds is preferred in the above dental care.

The present invention provides for the use of an effective amount of a salt, oxide or complex of one or more polyvalent metals to prepare a medicament (a medicinal substance or agent that treats or prevents or alleviates a medical condition such as a disease and/or its symptoms) useful for: preventing anaphthous ulcer on a mucosal surface of a mammal; treating anaphthous ulcer on a mucosal surface of a mammal wherein the medicament comprises a mucoadhesive paste or gel; treating one or more pre-ulceration symptoms of anaphthous ulcer on a mucosal surface of a mammal; treating a herpes simplex lesion on the skin of a mammal wherein the polyvalent metal is not zinc; or preventing a herpes simplex lesion (ulcer) on the skin of a mammal wherein the polyvalent metal is not zinc.

The present invention also provides a method for preventing anaphthous ulcer on a mucosal surface of a mammal comprising topically administering an effective amount of a salt, oxide or complex of one or more polyvalent metals to a mucosal surface demonstrating pre-ulceration symptoms so as to prevent the ulcer.

The present invention further provides a method for treating anaphthous ulcer on a mucosal surface of a mammal comprising topically administering an effective amount of a salt, oxide or complex of one or more polyvalent metals in a mucoadhesive paste to the ulcer.

The present invention also provides a method for treating one or more pre-ulceration symptoms of anaphthous ulcer on a mucosal surface of a mammal comprising topically administering an effective amount of a salt, oxide or complex of one or more polyvalent metals to a mucosal surface demonstrating pre-ulceration symptoms. Additionally, the present invention provides a method for treating a herpes simplex lesion on the skin of a mammal comprising topically administering an effective amount of a salt, oxide or complex of one or more polyvalent metals to a mammal in need of such treatment, where the polyvalent metal is not zinc.

There is also provided a method for preventing a herpes simplex lesion (ulcer) on the skin of a mammal comprising topically administering an effective amount of a salt, oxide or complex of one or more polyvalent metals to an area of skin demonstrating pre-lesion symptoms so as to prevent the lesion, where the polyvalent metal is not zinc.

The invention also provides a method for preventing anaphthous ulcer in a mammal comprising topically administering an effective amount of one or more anti-inflammatory compounds (steroidal and/or nonsteroidal) with or without an effective amount of a salt, oxide or complex of one or more polyvalent metals to the affected mucosal or skin surface likely to suffer from anaphthous ulcer. Addition of an anti-inflammatory compound such as hydrocortisone acetate to a polyvalent metal compound synergistically potentiates the activity of the metal compound and reduces the chance of flare-ups of lesion or ulcer. A novel ultra-low-strength of hydrocortisone or its derivative such as 0.02% is surprisingly found to be therapeutically highly effective (Example XVIII). Strengths of 1.0% and 2.5% (more than 100-fold higher than used in this invention) in ointments or creams are commercially available in the last several decades for external use for controlling itch and inflammation but not for aphthous ulcers.

The invention also provides the use of an effective amount of a salt, oxide or complex of one or more polyvalent metals alone, or more preferably with a combination of an anti-inflammatory compound, to prepare a medicament useful for: preventing or treating oral ulcers of a mammal resulting from chemotherapy or radiation therapy; or providing relief of pain, inflammation, and wound healing of burned tissue of a mammal, wherein the polyvalent metal is not silver.

The present invention also provides a method for the prophylaxis or treatment of oral ulcers of a mammal resulting from conventional chemotherapy and radiation therapy (e.g., cancer treatment therapy) comprising topically administering an effective amount of a salt, oxide or complex of one or more polyvalent metals, preferably with a combination of an anti-inflammatory compound, to a mucosal surface likely to suffer or suffering from ulcer formation.

The present invention also provides a method for providing relief of pain, inflammation, infection and the promotion of wound healing of burned tissue, as a result of fire or heat of a mammal comprising topically administering an effective amount of a salt, oxide or complex of one or more polyvalent metals, preferably with a combination of an anti-inflammatory compound, to the affected burned tissue wherein the polyvalent metal is not silver. When treating burn tissue, it is preferred that an effective amount (ranging from about 0.1% to about 2% preferred) of one or more polyvalent metals alone or preferably in combination with one or more anti-inflammatory compounds is administered in a solution, a liquid, a spray, or a gel, preferably containing at least about 20% by weight of glycerin, that will serve as an effective liquid protective layer for the burned tissue.

The therapeutic compositions and methods described herein provide the following benefits: (1) rapidly (within about 5 minutes to about 15 or about 20 minutes) inhibit or stop the tingling or burning sensation associated with an oral aphthous ulcer or an oral pre-ulcerous area of the mouth after a single application; (2) rapidly (within minutes) abolish the pain and irritation of an inflammatory red spot or erythematous bump (pre-ulceration symptoms) of the oral tissue after a single application; (3) completely cure the inflammatory red spot or erythematous bump (associated with pre-ulceration of the oral mucosa) of oral tissue after about one to about five applications and within about 24 hours from the beginning of treatment; (4) quickly (within minutes) stop the pain associated with an inflammatory aphthous ulcer after a single application; (5) completely cure/heal the ulcerated inflamed tissue of anaphthous ulcer after about one to about five applications of the medicament within about 12 hours to about 48 hours; (6) do not require a physician's prescription; (7) are virtually free of allergic reactions (e.g., hypoallergenic and/or nonallergenic); (8) are pharmacologically safe with minimum or no toxicity; (9) do not result in the development of resistance of microorganisms to antibiotics; (10) are relatively inexpensive; (11) are chemically stable; (12) are not distasteful or unpleasant to the taste (e.g, they are pleasant to taste); (13) are easy to apply and store; (14) may not contain a high-potency glucocorticoid steroid (e.g., a synthetic steroid with an anti-inflammatory potency that is many times more potent than hydrocortisone, or cortisone, both being endogenous, low-potency steroids); or (15) are able to simultaneously provide the needed analgesic, anti-inflammatory, anti-microbial and tissue-regenerating properties for controlling, preventing, or treating pre-ulceration and/or post-ulceration symptoms related to aphthous ulcers and/or herpes simplex lesions, as well as for treating and preventing gingivitis, periodontitis and tooth mobility, and for managing potential side effects resulting from chemotherapy and radiation therapy and for treating burned tissues.

### DETAILED DESCRIPTION

The present invention includes methods to quickly (within about 5 to about 20 minutes) abolish the pain associated with pre-ulceration (e.g., stinging or burning sensation, painful red spots, painful erythematous bumps and mouth injury) and post-ulceration conditions (e.g., painful and inflammatory lesions/ulcerated skin and/or ulcerated mucosal surfaces) of aphthous ulcers or herpes simplex lesions of a human after one application of a medicament comprising an effective amount of one or more salts or oxides of polyvalent metals; for aphthous ulcers and its pre-ulceration symptoms a combination with one or more safe and effective anti-inflammatory compounds is preferred because of the synergistic or additive effect. The present invention also includes methods to rapidly and completely (within about 12 hours to about 48 hours) heal or cure an inflamed aphthous ulcer. Therefore, compared to conventional drug therapy, methods of the present invention provide the hitherto most efficacious treatment and prevention (prophylaxis) of aphthous ulcers and symptoms associated therewith in the history of mankind. The medicaments of the invention include, for example, an effective amount of one or more polyvalent metal compounds, such as magnesium sulfate, aluminum sulfate or bismuth subsalicylate, alone or preferably with one or more anti-inflammatory compounds, such as hydrocortisone acetate and other esters. In one embodiment, the medicaments comprise a mucoadhesive agent to form a mucoadhesive paste. The medicaments and methods of the invention are also effective to treat and/or prevent herpes simplex lesions (see Example XVII), oral ulcers resulting from chemotherapy or radiation therapy (e.g., cancer treatment therapy), and to treat tissue that has been burned by fire or heat.

An extension of the present surprising invention for treating aphthous ulcers and herpes simplex is that polyvalent metal compounds are also useful in preventing and treating gingivitis, periodontitis and tooth mobility because of their very unique pharmacological properties disclosed herein (Examples V, VI and XX). A combination with one or more anti-inflammatory and/or tissue-regenerating compounds may be preferred for synergistic effects. Obviously, any other compound(s) that may enhance the therapeutic effect of various applications of the present invention can also be included in the medicament.

As used herein, the term “lesion” or “ulcer” includes lesions or ulcers of the lips and adjacent skin, the tongue, the gums and other internal tissues of the oral cavity, and the pharynx. The lesion or ulcer can be anaphthous ulcer, anaphthous-like ulcer, a lesion of the pharynx, or a lesion associated with herpes simplex virus, gingivitis, stomatitis, periodontitis or tooth mobility. The lesion or ulcer can be of the mucosal, submucosal, epidermal, dermal and/or subcutaneous tissue. The lesion can be a sore or rash related to the hand, foot and mouth disease.

“Treating,” as used herein, includes ameliorating the symptoms of, curing, and preventing (in a very broad definition) the development of a given disease or condition; a narrow definition is generally used in the present application. Preferably, the disease or condition to be treated or prevented is anaphthous ulcer, a herpes simplex lesion, gingivitis, periodontitis or tooth mobility. As used herein, the term “prevention” includes the prevention (i.e., prophylaxis) of ulceration in an area demonstrating pre-ulceration symptoms.

As used herein, the phrase “pre-ulceration symptom(s)” of aphthous ulcers refers to stinging, burning, pain, inflammation, red spots, erythematous bumps and mouth injury prior to the formation of ulcers in the mouth.

As used herein, the phrase “post-ulceration symptoms” refers to the pain, inflammation and ruptured tissue of the ulcer or lesion.

As used herein, the term “bioadhesive” refers to a substance, such as a paste or biologically compatible cement, that provides or promotes adhesion to a biological surface. The term “mucoadhesive” refers to a substance, such as a paste or biologically compatible cement, which provides or promotes adhesion to mucosal surfaces. Bioadhesive and mucoadhesive agents include, but are not limited to, carboxymethylcellulose or derivatives thereof, carbomer, xanthan gum, ULTREX-10 (a polyacrylic acid polymer used as a thickening, bioadhesive or mucoadhesive agent), polyacrylic acid polymer, gelatin, chitosans, hyaluronates, or a mixture thereof. The bioadhesive substance and/or the mucoadhesive substance will allow for prolonged (about 0.5 to about 8 hours or longer) direct contact between the tissue (biological surface) and the medicinal composition, and thus, allow for a continuous and effective delivery system.

As used herein, the phrase “thickening agent” refers to any substance/material used to thicken a medicinal composition. Thickening agents include, but are not limited to, carboxymethylcellulose or derivatives thereof, veegum, carbomer, polyacrylic acid polymer, gelatin or a mixture thereof.

The phrase “effective amount” refers to that amount of a compound that is sufficient to effect safe treatment when administered to a mammal in need of such treatment or prevention.

As used herein, the phrase “a polyvalent metal compound” refers to any organic or inorganic polyvalent metal compound that has the beneficial therapeutic and safe properties described herein. Polyvalent metal compounds include bismuth compounds, zinc compounds, magnesium compounds, aluminum compounds, calcium compounds, titanium compounds, iron compounds, copper compounds, manganese compounds, chromium compounds, selenium compounds, molybdenum compound, or a barium compounds. In one embodiment of the invention, the polyvalent metal is not aluminum, zinc or silver. In another embodiment of the invention, the polyvalent metal is not a Group IIIa element, such as boron, aluminum, gallium, indium, or thallium. Clearly, various inventions described in this application will reduce suffering and provide comfort to patients.

Applicant has surprisingly discovered that polyvalent metals have beneficial properties including analgesic, anti-inflammatory, anti-microbial and tissue-regenerating properties for controlling, preventing, or treating pre-ulceration and/or post-ulceration symptoms related to aphthous ulcers and/or herpes simplex lesions as well as for treating gingivitis, periodontitis and tooth mobility. Thus, according to the methods of the invention, an amount of a polyvalent metal effective to produce the stated therapeutic activity is administered. When a polyvalent metal salt is administered according to a method of the invention, preferably the counterion to the metal can also contribute significantly to the stated therapeutic effect; magnesium salicylate and bismuth salicylate are such examples because the salicylate has an anti-inflammatory and analgesic activity; any polyvalent metal oxide, salt or complex known to date to have the described therapeutic activity is excluded from the current invention. In one embodiment of the invention, the “polyvalent metal salt” is an inorganic or organic salt, oxide or complex. In another embodiment of the invention, the “polyvalent metal salt” is an inorganic salt or an organic compound. Both water-soluble (solubility greater than 1 in 50) polyvalent metal compounds, such as magnesium sulfate and ferric chloride, and water-insoluble (solubility less than 1 in 1,000) polyvalent metal compounds, such as bismuth citrate and magnesium hydroxide, are therapeutically effective (see Examples). Thus, both ionic and nonionic species of the polyvalent metals are therapeutically effective.

The polyvalent metal compounds useful in this invention include bismuth compounds, zinc compounds, magnesium compounds, aluminum compounds, calcium compounds, titanium compounds, iron compounds, copper compounds, molybdenum compounds and barium compounds; silver compounds are not included in this invention. Complete listing of these compounds in this application is clearly not possible. The following cited examples are mainly obtained from the following references: (1) “The Merk Index,” Merck & Co., Inc., Whitehouse Station, New Jersey, 2001, and (2) “Martindale, The Extra Pharmacopeia,” or “Martindale, The Complete Drug Reference,” edited by J. E. F. Reynolds, The Pharmaceutical Press, London, 1999. Complexes of a polyvalent metal compound with various amino acids are also potential candidates in this invention.

In one embodiment of the invention, the therapeutically effective compound is selected from the group consisting of bismuth, bismuth subsalicylate, bismuth chloride, bismuth oxide, bismuth subcarbonate, bismuth subgallate, bismuth subnitrate, bismuth phosphate, bismuth aluminate, bismuth salicylate, bismuth tribromophenate, bismuth dipropylacetate, bismuth citrate, bismuth subcitrate, bismuth ascorbate, bismuth subcarbonate, bismuth tartrate, and colloidal bismuth subcitrate, and other bismuth-containing compounds.

Additionally, the active ingredient in Pepto-Bismol® (bismuth subsalicylate) and Bismatrol® (bismuth subsalicylate) is bismuth subsalicylate. Thus, Pepto-Bismol® and Bismatrol® are examples of compositions that comprise a bismuth compound.

In another embodiment of the invention, the therapeutically effective compound is selected from the group consisting of zinc, zinc sulfate, zinc acetate, zinc gluconate, zinc chloride, zinc carbonate, zinc oxide, zinc oleate, zinc stearate, zinc propionate, zinc ascorbate, zinc undecenoate, zinc salicylate and other zinc-containing compounds.

In another embodiment of the invention, the therapeutically effective compound is selected from the group consisting of magnesium, magnesium acetate, magnesium ascorbate, magnesium carbonate, magnesium chloride, magnesium citrate, magnesium stearate, magnesium gluconate, magnesium hydroxide, magnesium salicylate, magnesium sulfate, magnesium oxide, magnesium ascorbate, magnesium valproate, magnesium asparate and other magnesium-containing compounds.

In another embodiment of the invention, the therapeutically effective compound is selected from the group consisting of aluminum, aluminum acetate, aluminum carbonate, aluminum chloride, aluminum potassium sulfate, aluminum glycinate, aluminum hydroxide, aluminum lactate, aluminum oxide, aluminum subacetate, aluminum sulfate, aluminum phosphate, aluminum ascorbate, aluminum citrate, aluminum salicylate, aluminum aspirin, aluminum glycyrrhetinate and other aluminum-containing compounds.

In another embodiment of the invention, the therapeutically effective compound is selected from the group consisting of calcium, calcium acetate, calcium alginate, calcium benzoate, calcium carbonate, calcium chloride, calcium citrate, calcium gluconate, calcium hydroxide, calcium lactate, calcium phosphate, calcium stearate, calcium sulfate, calcium oxide, calcium ascorbate, calcium salicylate and other calcium-containing compounds.

In another embodiment of the invention, the therapeutically effective compound is selected from the group consisting of copper, copper gluconate, copper sulfate, copper ascorbate, and other copper-containing compounds.

In one embodiment of the invention, the therapeutically effective compound is selected from the group consisting of titanium, titanium dioxide, titanium peroxide, titanium salicylate, titanium tannate, titanium ascorbate and other titanium-containing compounds.

In another embodiment of the invention, the therapeutically effective compound is selected from the group consisting of iron, ferric chloride, ferric citrate, ferric oxide, ferrous ascorbate, ferrous carbonate, ferrous sulfate, ferrous gluconate, ferrous fumarate, ferrous glycine sulfate, ferrous lactate, and other iron-containing compounds.

In another embodiment of the invention, the therapeutically effective compound is selected from the group consisting of barium, barium carbonate, barium chloride, barium hydroxide, barium sulfate, barium ascorbate and other barium-containing compounds.

In a preferred embodiment of the invention, the polyvalent metal compound is magnesium (e.g., magnesium sulfate or magnesium chloride). Applicant has found magnesium to be very effective in treating and preventing aphthous ulcers or herpes simplex lesions, in treating pre-ulceration or pre-lesion symptoms, such as pain and inflammation, and in treating post-ulceration or post-lesion symptoms, and in treating gingivitis, periodontitis, and tooth mobility. Additionally, magnesium possesses many preferred characteristics for polyvalent metals for use in the present invention, such as pleasant to taste, colorless, odorless, endogenous, relatively non-toxic, relatively non-irritating, inexpensive, and stable.

The polyvalent metal compounds can be formulated as pharmaceutical compositions and administered topically to a human patient in a variety of forms, including but not limiting to gels, ointments, creams, pastes, lotions, liquids (e.g., oral rinses such as mouth washes/rinses), a lozenge, a medicated bandage, a spray or any other form which is suitable for topical and effective administration to an oral or percutaneous ulcer/lesion. Incorporation of magnesium compounds such as magnesium sulfate in tooth paste or in mouth wash is useful for promoting health of the oral mucous membranes and tissues and for reducing the dental diseases described in this invention when it is applied daily or regularly.

For topical administration, the polyvalent metal compounds may be applied in pure form. However, it will generally be desirable to administer them to the mucosal surface or skin as compositions or formulations in combination with a suitable dermatologically or pharmaceutically acceptable carrier, which may be a solid or a liquid. A suitable dermatologically or pharmaceutically acceptable carrier includes any dermatologically or pharmaceutically acceptable carrier which is capable of mixture with a polyvalent metal compound.

Useful solid carriers include carboxymethylcellulose or derivatives thereof, carbomer, polyacrylic acid polymer, veegum, gelatin or a mixture thereof. Useful liquid carriers include water, glycerin, alcohols or glycols or water-alcohol/glycol blends, in which the present compounds can be dissolved or dispersed at effective levels, optionally with the aid of non-toxic surfactants. The resultant liquid compositions can be applied from absorbent pads, as a liquid “film” (a film formed after the solvent evaporates) used to impregnate bandages and other dressings, sprayed onto the affected area using pump-type or aerosol sprayers, or used as mouth rinses.

In a preferred embodiment, the medicament which is administered to a patient comprises a polyvalent metal compound and a bioadhesive or mucoadhesive paste. Generally, dosage forms that comprise bioadhesives and/or mucoadhesives provide prolonged (about 0.5 hour to about 8 hours or longer) direct contact with the damaged and/or diseased (e.g., pre-ulcerous and/or post-ulcerous) tissue area.

Drying of the tissue area of lesion with a clean paper, tissue, cotton tip, or gauze before administration of a medicament of the invention can facilitate close contact, maintain high drug concentrations at the lesion site and hence enhance the efficacy of the treatment. In order to minimize dilution of medicaments by the saliva continuously formed in the mouth cavity and to achieve the maximum therapeutic benefit, after topical administration, patients should have the mouth open slightly in a lying down, sitting or standing position. With the mouth open, the saliva produced can more easily flow directly into the stomach without accumulation in the mouth cavity. Administration of medicament shortly before going to bed is preferred because a more prolonged contact between the drug and the lesion can result during sleep. Additionally, even though the patient may be symptom free by the following morning, the patient should continue topical application of the medicament after breakfast and again after lunch to reduce the possibility of “flare-ups” during the second day as a result of the vulnerability of freshly-recovered lesion to “stress” from the day. Using a combination of a polyvalent metal compound and an anti-inflammatory compound reduces the chance of the flare-ups for pre-ulceration and post-ulceration symptoms of aphthous ulcers. In general, such a synergistic combination is recommended for various applications disclosed in this invention. Patients should avoid eating, drinking and talking after medication as long as possible (for example, about one hour) due to a possibility of washing or moving the drug away from the administration site. Protection of medicament in the mouth by inserting a piece of gauze may be valuable under certain circumstances. During or shortly after treatment, the patient should avoid eating foods or doing activities known to provoke the disease or symptoms.

When using a liquid mouth rinse/wash, the patient should first swirl the medicament in the mouth for several seconds (to ascertain contact between the medicament and the surface of the lesion) and hold the medicament in the mouth for as long as possible (from a few minutes to about 30 minutes) by closing the mouth. After spitting out the diluted medicament, the patient should not drink or eat for about 30 to about 60 minutes to allow a longer time of contact between the medicament and the surface of the lesion in the mouth. To enhance the contact time between the medicament and the surface lesion, a viscosity-enhancing agent, such as xanthan gum, glycerin and carboxymethylcellulose or a derivative thereof, may also be incorporated into the mouth rinse, even making it like a paste (swallow being allowed). A fragrance, coloring agent, a sweetening agent and/or a preservative may also be added to the preparation.

Generally, the concentration of a polyvalent metal compound in a liquid, semi-solid, or solid composition will be about 0.01% to about 50%, preferably about 0.1% to about 10%, more preferably about 0.01% to about 3.5%, more preferably 0.03% to about 2.5%, and more preferably about 0.05% to about 2.0% weight by volume. In a preferred embodiment the polyvalent metal compound(s) is present in the pharmaceutical compositions/medicaments in amounts ranging from about 0.01% to about 30%, preferably from about 0.02% to about 3% weight by volume.

In a preferred embodiment an anti-inflammatory compound is administered in combination with a polyvalent metal compound. Anti-inflammatory compounds include glucocorticoidal steroids such as hydrocortisone, cortisone, prednisolone, dexamethasone, or triamcinolone acetonide, as well as nonsteroidal anti-inflammatory drugs (NSAIDs), such as salicylate, ibuprofen, naproxen, acetaminophen, indomethocin, ketoprofen or glycyrrhizin. In one embodiment, the anti-inflammatory compound is an endogenous glucocorticoid or its derivative (such as ester); they include, for example, hydrocortisone, hydrocortisone acetate, hydrocortisone butylate, hydrocortisone cipionate, hydrocortisone valerate, hydrocortisone sodium phosphate or succinate, hydrocortisone glycyrrhetinate, cortisone and cortisone acetate. Other endogenous glucocorticoidal steroids and their derivatives may also be used. The inclusion of an anti-inflammatory compound markedly enhances the therapeutic activity, including the prevention and treatment of pre- and post ulceration symptoms and conditions, of the polyvalent metal compounds. Effective anti-inflammatory compounds without the need of a prescription are preferred.

Additionally, topical administration of nonsteroidal anti-inflammatory compounds and/or low-potency glucocorticoidal steroids, such as hydrocortisone, hydrocortisone acetate and other derivatives, cortisone or cortisone acetate and other derivatives, without the addition of a polyvalent metal compound is also beneficial in preventing aphthous ulcers, in treating their pre-ulceration symptoms and in treating ulcers. Generally, the concentration of an anti-inflammatory compound in a liquid, semi-solid, or solid composition will be about 0.001% to about 5%, more preferably about 0.05% to about 1.0% weight by volume.

It is understood that the meaning of the phrase “an effective amount of one or more polyvalent metal compounds” is an amount of one or more polyvalent metal compounds that is effective to reduce or diminish the size of the lesion, ulcer and/or inflamed tissue area, shorten the time for relief from the symptoms, and/or cause a reduction in the symptoms of aphthous ulcers, herpes simplex lesions, gingivitis, periodontitis or tooth mobility. For instance, the methods of the present invention relieve pre-ulceration and pre-lesion symptoms as wells as ulcers, lesions, post-ulceration and post-lesion symptoms and conditions, including pain, inflammation, irritation, and discomfort, within about 5 to about 20 minutes after topical administration of the polyvalent metal compound(s) to the affected tissue. Additionally, the methods of the present invention heal or cause the ulcer, lesion or inflammatory bump to disappear within about 5 hours or about 8 hours to about 48 hours after topical administration of the polyvalent metal compound. This is in very sharp contrast with conventional drug therapy. Resolution of pain and healing of aphthous ulcers and/or herpes simplex lesions by conventional methods of treatment generally requires several days of continuous (3-to-4-times-a-day) treatment.

It is emphasized that the surprising and unexpected efficacy of polyvalent metal compounds for the prophylaxis, treatment and/or cure of aphthous ulcers, herpes simplex lesions, gingivitis, periodontitis and tooth mobility described herein can be attributed to the hitherto unreported combined surprising effects of their analgesic, anti-inflammatory, anti-microbial and tissue-regenerating properties.

The invention will now be illustrated by the following non-limiting Examples.

### Example I

### Prophylaxis and Treatment of Aphthous Ulcers Using Pepto-Bismol (Bismuth Subsalicylate)

Pepto-Bismol® (Proctor and Gamble, Cincinnati, Ohio) is a commercial nonprescription suspension product containing 262 mg of bismuth subsalicylate per tablespoon (15 ml) as the active ingredient (i.e., 1.75%). The product label for Pepto-Bismol® states that its recommended use is for the treatment of upset stomach, indigestion, nausea, heartburn and diarrhea. The normal dose for adults is 30 ml, which may be repeated every 0.5 to 1.0 hour for a total of 8 doses (i.e., 240 ml) in one day.

Prior to bedtime, a small amount of Pepto-Bismol®, which had been concentrated through evaporation, was administered several times to the surface area of a painful aphthous ulcer (about 0.30 cm in size), which developed under a human tongue three days earlier. The following morning, there was no longer any pain associated with the lesion.

The “concentrated” Pepto-Bismol® was administered twice more that day, after which, the lesion was healed. The concentrated Pepto-Bismol® suspension has also been used to quickly reverse further development of the early (no visible exudate) stages of aphthous ulcers.

### Example II

### Treatment of Pre-Ulceration Symptoms and Prophylaxis of Aphthous Ulcers Using Bismuth Citrate Oral Paste A (Gel)

A water-based oral paste A containing 1% bismuth citrate as an active ingredient was prepared. The paste also contained about 30% glycerin, 1% sodium bicarbonate, 1% Carbopol® ULTREX-10 (a polyacrylic acid polymer used as a thickening, bioadhesive or mucoadhesive agent) and 63% water. The paste was administered to two inflammatory, painful bumps around the tongue and to a painful lesion on the floor of the oral cavity after drying these areas with clean tissue paper before bedtime. The pain literally stopped in a few minutes, and almost complete healing was noted the next morning.

### Example III

### Treatment of Pre-Ulceration Symptoms and Prophylaxis of Aphthous Ulcers Using Bismuth Citrate and Naproxen Oral Paste A

A water-based paste containing 1% bismuth citrate and 0.5% sodium naproxen with sodium carboxymethylcellulose as a thickening agent was prepared. A small amount of this paste was administered before bedtime to two aphthous ulcers of the tongue at the onset of development. The following morning, there were no longer any symptoms of the disease, and no further administration was needed. This novel treatment regimen was repeated successfully two more times.

### Example IV

### Treatment of Aphthous Ulcer Using Bismuth Citrate/Hydrocortisone Oral Paste A

A water-based oral paste containing about 0.6% bismuth citrate, 0.8% hydrocortisone, 27% glycerin, 61% water and 11% Carbopol® ULTREX-10 was prepared. Prior to bedtime, the area surrounding a small aphthous ulcer on the tip of a human tongue was dried with a cotton tip. Next, a small amount of the oral paste was administered to the lesion and the surrounding area. The following morning, there were no longer any symptoms of the disease.

### Example V

### Treatment of Gingivitis Using Bismuth Citrate/Hydrocortisone Oral Paste A

Prior to bedtime, a small amount of the bismuth citrate/hydrocortisone oral paste A described in Example IV was administered to a swollen, painful to the touch, magenta gingivitis developed under two human teeth of the upper-left, front gum (the affected area was dried with cotton tip or ball prior to administration of the paste). The following morning, there was substantial improvement in the symptoms. The symptoms of the gingivitis no longer existed after administration of the oral paste two to three times a day for four days. A stainless steel spatula was used to assist in the administration of the paste.

### Example VI

### Treatment of Ulcerative Gingivitis Using Bismuth Citrate/Hydrocortisone Oral Paste A

Upon the development of a gingival ulcer with diffuse bleeding in the lower left gum of a human mouth, the oral paste, as described in Example IV, was administered prior to bedtime. The next morning, the gum was no longer bleeding.

After further administration (twice a day for two days), the redness of the surrounding gingiva disappeared, the color returned to normal, and the size of the lesion was found to be approximately reduced by half (from about 0.4 mm to about 0.2 mm in length). Furthermore, after administration of the oral paste for three more days there was no pain or bleeding after probing the affected area with a toothpick. Additionally, the depth of the lesion appeared to be markedly reduced, and the affected area looked much healthier. Administration of medicament was facilitated by using a stainless spatula.

### Example VII

### Prophylaxis and Treatment of Aphthous Ulcers with Bismuth Citrate/Hydrocortisone Oral Paste B

A lower strength of bismuth citrate (0.2%) and hydrocortisone (0.2%) oral paste B was also prepared similar to that described in Example II. A small amount of oral paste B was administered to anaphthous ulcer on the tip of a human tongue, after drying the affected lesion area with tissue paper, prior to bed. The pain stopped almost immediately. The next morning, the ulcer, and its associated pain, were no longer present.

A single overnight administration of oral paste B was also successful to prevent the full development of anaphthous ulcer in a human. Upon the sensation of a mild stinging pain, an early sign of anaphthous ulcer, on the tip of a human tongue, oral paste B was administered overnight. In the morning, the stinging sensation no longer existed and the patient did not develop a full aphthous ulcer. Thus, the early administration of medicaments of the present invention may effectively prevent a later painful full-blown development of aphthous ulcers.

### Example VIII

### Treatment of Gingivitis with Bismuth Citrate/Hydrocortisone Oral Paste B

Oral paste B was administered two times a day to an area of gingivitis in a human for three days. After administration of this treatment, the gingival color returned to normal and the area no longer bled upon probing.

### Example IX

### Prophylaxis and Treatment of Aphthous Ulcers with Aluminum Hydroxide/Magnesium Hydroxide Oral Paste C

Oral paste C containing 0.25% aluminum hydroxide and 0.25% magnesium hydroxide was prepared similar to that of above oral paste B. After drying the tip of a human tongue with clean tissue paper, a small amount of oral paste C was administered to an area of demonstrating early signs (pre-ulcer stage) of a canker sore area before going to bed. The pain disappeared in a few minutes after administration. The inflammatory bump disappeared by the next morning. Similar results were obtained on other occasions.

### Example X

### Treatment of Aphthous Ulcer with Zinc Acetate Oral Paste D

Oral paste D containing 0.25% zinc acetate was prepared similar to that of oral paste B. A stinging pain with a small ulcer was noticed under the tongue of a human subject. At midnight, after drying the area with a clean paper tissue, a small amount of oral paste D was administered to the lesion and the pain stopped almost immediately. No pain was felt and the lesion was practically healed next morning. It is to be noted that administration of a placebo oral paste had no effect on relieving pain of the canker sore.

### Example XI

### Treatment of Pre-Ulcerous Symptoms of Aphthous Ulcers with Aluminum Potassium Sulfate Oral Paste E

Oral paste E containing about 0.25% aluminum potassium sulfate (i.e., alum) was prepared similar to oral paste B. The pH was adjusted to 4.8 with sodium carbonate. A painful bump (early stage of a canker sore) was noted in the right front edge of the tongue of a human subject. Oral paste E was administered to the affected area before bedtime. The pain from the pre-ulcerous symptom stopped within a few minutes after administration of Oral paste E. Next morning, the paste was administered two more times. The bump and pain disappeared before noon.

### Example XII

### Treatment of Painful Bumps of Aphthous Ulcers with Magnesium Sulfate Oral Paste F

Oral paste F containing 0.5% anhydrous magnesium sulfate was similarly prepared as oral paste E. A sizable red bump (pain upon touch) on the tongue of a human subject was treated with oral paste F before bedtime. The bump disappeared by the next morning. Similar pastes containing 1% or 4% magnesium sulfate were also found to be similarly effective after one application prior to bedtime.

### Example XIII

### Treatment of Pre-Ulceration Symptoms of Aphthous Ulcer with Ferric Citrate Oral Paste G

Oral paste G containing 0.15% ferric citrate was similarly prepared as oral paste E. A single administration of oral paste G before bedtime was found to effectively treat the bump on a tongue on two occasions in a human subject.

### Example XIV

### Treatment of Painful Aphthous Ulcers with Magnesium Sulfate Oral Paste H

A 4.0% magnesium sulfate paste (the pH was adjusted to about 6.0 with sodium hydroxide) with Carbopol® 974P as a mucoadhesive agent was prepared and applied to an about 0.5 cm size of aphthous ulcer on left topside of the tongue in a human subject before bedtime. The pain disappeared in about 15 minutes and no pain was felt the next morning. After eating breakfast, the paste was applied again and the aphthous ulcer was completely healed after the second application.

### Example XV

### Treatment of Painful Aphthous Ulcers and Erythematous Bumps with Magnesium Sulfate and Hydrocortisone Acetate Oral Pastes

Oral paste containing 3.0% magnesium sulfate and 0.2% hydrocortisone acetate with Carbopol 974P (BF Goodrich, Cleveland, Ohio) was prepared. A human patient developed two painful medium-sized aphthous ulcers, one on the top surface of the tongue and the other underneath the tongue. The pain severely affected the eating and talking capabilities of the patient. The oral paste was applied before dinner to both ulcerous areas. The pain completely disappeared within about 20 minutes and the patient was able to talk and eat throughout dinner without any pain. The paste was applied again prior to bedtime. Both ulcers were found to be reduced in size the next morning and there was no pain. The paste was applied again after breakfast, after lunch and prior to bed. No pain was felt through the day and both ulcers were found to be completely healed/cured the next morning. No “flare up” or recurrence of the aphthous ulcer developed.

The paste was also used to completely heal a small erythematous (e.g., redness of the mucosal tissue of the oral cavity caused by dilatation and congestion of the capillaries) bump with only one application prior to bed.

An additional paste containing 1.5% magnesium sulfate and 0.05% hydrocortisone acetate using xanthan gum as the thickening agent was also successfully used to treat/prevent aphthous ulcers in 4 adults with 100% curing rate in one to two days.

The safety of the above paste is obvious from the fact that both magnesium and hydrocortisone are endogenous substances and the amounts of the drugs present in the paste is minute compared to those normally present in the human body.

### Example XVI

### Treatment of Irritating and Inflammatory Bumps with a 2.5% Magnesium Sulfate Mouth Rinse

A human subject had two small irritating inflammatory bumps (pre-ulcerous symptom) at the tip of the tongue for about two days. Approximately 30 minutes prior to bedtime, the subject rinsed his mouth by swirling about 10 ml of an aqueous 2.5% magnesium sulfate solution for about 10 seconds and holding it in his mouth for about 8 minutes prior to spitting it out. This mouth rinse was repeated two more times prior to bedtime. After about 7 hours of sleep, the two bumps completely disappeared and no flare-up or recurrence occurred. Thus, the inflammatory and irritating bumps were completely healed/cured after 3 mouth rinses prior to bedtime. The mouth rinse was prepared by dissolving 2.5 grams of anhydrous magnesium sulfate in 100 ml of distilled water.

### Example XVII

### Treatment of Herpes Simplex Lesions

A paste containing 0.5% magnesium sulfate, 1% alum (aluminum potassium sulfate) and 0.2% bismuth citrate was similarly prepared. The paste was applied two to three times a day to a herpes simplex lesion near the tip of a human subject. After application, no pain was noticed and the lesion completely healed in about 3 days. A solution containing 15% alum, 10% water and 75% glycerin was applied to an inflammatory weeping lesion on the lip of another human subject. Very dramatic healing was found to begin the next day.

### Example XVIII

### Treatment of Pre-Ulceration Symptoms and Prophylaxis of Aphthous Ulcers with a Novel Ultra-Low-Strength Hydrocortisone or Hydrocortisone Acetate Paste

A paste containing 0.02% of hydrocortisone or hydrocortisone acetate using Carbopol® as a mucoadhesive agent was also similarly prepared. In one patient, these pastes have been repeatedly demonstrated to almost immediately (within about 10 to 20 minutes) and completely relieve the pain of inflammatory bumps or lesions after one single application before bedtime. Furthermore, the bumps were found to disappear next day following one additional application in the morning. No development of more serious aphthous ulcers occurred later. The strength of the steroid used above is much less than that (usually 0.1%) of high-potency steroids, such as triamcinolone acetonide, used conventionally; the strength employed is regarded as the “subtherapeutic” strength based on conventional steroid therapy.

### Example XIX

### Surprising Result of Treatment of Percutaneous Fissure or Crack with Magnesium Sulfate Paste

A human subject had a percutaneous fissure or crack (about 1 cm long and about 0.2 cm wide) in the lower leg. A 1% magnesium sulfate paste was applied to the fissure and covered with a bandage before bedtime. The pain completely disappeared and the fissure was completely healed (re-epithelialization) by the morning.

### Example XX

### Treatment for Gingivitis, Periodontitis and Tooth Mobility

An adult developed gingivitis with bleeding upon applying pressure. Pepto-Bismol® was applied two to three times a day to the area of lesion with a cotton ball that was placed between two neighboring teeth overnight. After several days of treatment, the bleeding stopped and the gum appeared to have a significant improvement.

In another adult with a chronic periodontitis (dull pain felt upon application of pressure or biting the teeth or eating) and a severe tooth mobility, an aqueous gel containing 1.5% of magnesium sulfate was applied to the lesion several times a day. Dull pain completely disappeared in about one week and tooth mobility improved significantly after about two weeks of treatment, presumably due to some tissue re-generation (see Example XIX) and elimination of infection by magnesium sulfate; the gum also appeared much healthier. On another occasion, a minor tooth tenderness was completely relieved after two applications. In another subject, periodontitis and tooth mobility were significantly improved by daily application of the gel for about one week.

It is to be understood that the above descriptions are intended to be illustrative, and not restrictive. Many other equivalents will be apparent to those of skill in the art upon reading and understanding the above description. Additionally, one skilled in the art will be able to ascertain, with no more than routine experimentation, many equivalents to the specific embodiments described herein. These equivalents are intended to be encompassed by the following claims.

All publications, patents, and patent documents are incorporated by reference herein, as though individually incorporated by reference.

1. A method of
a) preventing anaphthous ulcer on a mucosal surface of a mammal;
b) treating anaphthous ulcer on a mucosal surface of a mammal;
c) treating one or more pre-ulceration symptoms of anaphthous ulcer on a mucosal surface of a mammal;
d) treating a herpes simplex lesion on the skin of a mammal wherein the polyvalent metal is not zinc; or
e) preventing a herpes simplex lesion on the skin of mammal wherein the polyvalent metal is not zinc
comprising administering to said mammal an effective and safe amount of a salt, oxide or complex of one or more polyvalent metals in a suitable topical composition, wherein a comfort is provided to the mammal.

2. The method of claim 1, wherein the salt, oxide or complex is a bismuth compound, zinc compound, magnesium compound, aluminum compound, calcium compound, molybdenum compound, titanium compound, iron compound, copper compound, selenium compound, manganese compound, chromium compound or barium compound.

3. The method of claim 2, wherein the magnesium compound is selected from magnesium, magnesium acetate, magnesium ascorbate, magnesium carbonate, magnesium chloride, magnesium citrate, magnesium stearate, magnesium gluconate, magnesium hydroxide, magnesium salicylate, magnesium sulfate, magnesium oxide, magnesium valproate, magnesium alginate, magnesium asparate, and from other magnesium-containing compounds.

4. The method of claim 2, wherein the aluminum compound is selected from aluminum, aluminum acetate, aluminum carbonate, aluminum chloride, aluminum potassium sulfate, aluminum glycinate, aluminum hydroxide, aluminum lactate, aluminum oxide, aluminum subacetate, aluminum sulfate, aluminum phosphate, aluminum ascorbate, aluminum aspirin, aluminum glycyrrhetinate, aluminum citrate and from other aluminum-containing compounds.

5. The method of claim 2, where the bismuth compound is selected from bismuth, bismuth subsalicylate, bismuth chloride, bismuth oxide, bismuth subcarbonate, bismuth subgallate, bismuth subnitrate, bismuth phosphate, bismuth aluminate, bismuth salicylate, bismuth tribromophenate, bismuth dipropylacetate, bismuth citrate, bismuth subcitrate, bismuth ascorbate, bismuth subcarbonate, bismuth tartrate, colloidal bismuth subcitrate, and from other bismuth-containing compounds.

6. The method of claim 1, comprising administering a mucoadhesive agent in direct contact between the polyvalent metal and the mucosal surface for about 0.5 hour to about 8 hours or longer after administration of the polyvalent metal.

7. The method of claim 1, wherein the composition further comprises one or more anti-inflammatory compounds for providing relief of pain and inflammation and the healing of lesion.

8. The method of claim 7, wherein the anti-inflammatory compound is a glucocorticoid steroid.

9. The method of claim 8, wherein the glucocorticoid steroid is an endogenous compound or a derivative thereof.

10. The method of claim 9, wherein the endogenous compound or a derivative thereof is selected from hydrocortisone, hydrocortisone acetate, hydrocortisone butylate, hydrocortisone valerate, hydrocortisone glycyrrhetinate, hydrocortisone cipionate, hydrocortisone sodium phosphate or succinate, cortisone, cortisone acetate and other derivatives of hydrocortisone or cortisone.

11. The method of claim 7, wherein the anti-inflammatory compound is a non-steroidal anti-inflammatory drug.

12. The method of claim 1, wherein the metal compound comprises about 0.01% to about 50% weight by volume of the composition.

13. The method of claim 1, wherein the composition is useful for relieving pre-ulceration or pre-lesion symptoms within about 5 minutes to about 20 minutes after topical administration of the composition to the mucosal or skin surface.

14. The method of claim 1, wherein the composition is useful for healing the pre-ulceration symptoms within about 5 hours to about 24 hours after topical administration of the composition to the mucosal or skin surface.

15. The method of claim 1, wherein the composition is useful for relieving pain, inflammation or irritation associated with the ulcer or lesion within about 5 minutes to about 20 minutes after administration of the composition.

16. The method of claim 1, wherein the composition is useful for healing a lesion or ulcer within about 8 hours to about 48 hours after administration of the composition.

17. The method of claim 1, wherein the composition is suitable for administration one or more times.

18. A method for treating one or more pre-ulceration symptoms of anaphthous ulcer or preventing and treating anaphthous ulcer on a mucosal surface of a mammal comprising topically administering a composition comprising an effective and safe amount of salt, oxide or complex of one or more polyvalent metal compounds and an effective and safe amount of one or more anti-inflammatory compounds, wherein comfort to the mammal is provided.

19. The method of claim 18, wherein the polyvalent metal compounds are selected from bismuth, aluminum, magnesium, barium, iron, zinc, copper, titanium, molybdenum, selenium, manganese, chromium and calcium compounds and the anti-inflammatory compounds are selected from glucocorticoidal steroids and non-steroidal anti-inflammatory compounds.

20. The method of claim 19, wherein the anti-inflammatory compound is selected from a group of endogenous glucocorticoidal steroids and their derivatives with effective concentrations as low as about 0.001% to about 0.02% w/v.

21. A method for treating one or more pre-ulceration symptoms of anaphthous ulcer on a mucosal surface of a mammal comprising topically administering an effective and safe amount of a salt, oxide or complex of one or more polyvalent metals to a mucosal surface demonstrating pre-ulceration symptoms.

22. A method for treating a herpes simplex lesion on the skin of a mammal comprising topically administering an effective and safe amount of a salt, oxide or complex of one or more polyvalent metals alone or in combination with a safe and effective anti-inflammatory compound to a mammal in need of such treatment, where the polyvalent metal is not zinc.

23. A method for preventing a herpes simplex lesion on the skin of a mammal comprising topically administering an effective and safe amount of a salt, oxide or complex of one or more polyvalent metals alone or in combination with a safe and effective anti-inflammatory compound to an area of skin demonstrating pre-lesion symptoms so as to prevent the lesion, where the polyvalent metal is not zinc, wherein comfort to the mammal is provided.

24. A method of
a) preventing or treating oral ulcers of a mammal resulting from chemotherapy or radiation therapy; or
b) providing relief of pain and healing of burned tissue of a mammal
comprising topically administering to said mammal an effective and safe amount of a salt, oxide or complex of one or more polyvalent metals alone or in combination with one or more safe and effective anti-inflammatory compounds.

25. A method for the prophylaxis or treatment of oral ulcers of a mammal resulting from chemotherapy or radiation therapy comprising topically administering an effective and safe amount of a salt, oxide or complex of one or more polyvalent metals alone, or in combination with an effective and safe anti-inflammatory compound to a mucosal surface likely to suffer or suffering from ulcer formation.

26. A method for providing relief of pain and promotion of wound healing of burned tissue of a mammal comprising topically administering an effective and safe amount of a salt, oxide or complex of one or more polyvalent metals alone, or in combination with an effective and safe anti-inflammatory compound to the affected burned tissue.

27. The method of claim 26, wherein the salt, oxide or complex is formulated as a solution, a liquid, a spray, or a gel.

28. The method of claim 27, further comprising at least about 20% or more by weight glycerin.

29. A method for providing prophylaxis and treatment of gingivitis, periodontitis and tooth mobility of a mammal comprising topically administering in a proper dosage form an effective and safe amount of a salt, oxide or complex of one or more polyvalent metal compounds alone, or in combination with an effective and safe amount of one or more anti-inflammatory compounds to the site of lesion, wherein comfort is provided to the mammal.

30. The method of claim 29, wherein the polyvalent metal is magnesium, aluminum, bismuth, calcium or zinc.

31. A method for promoting health of oral mucous membranes and tissues and for treating gingivitis, periodontitis and tooth mobility of a mammal comprising topically administering in a proper dosage form an effective and safe amount of a salt, oxide or complex of magnesium to the site of lesion or oral cavity, wherein the dosage form includes a gel, paste, ointment, suspension, liquid mixture, powder, spray or toothpaste.
